(19) United States
(12) Patent Application Publication
(10) Pub. No.: US 2017/0347890 A1
(43) Pub. Date: Dec. 7, 2017

(54) WEARABLE OCULAR SURFACE TEMPERATURE MONITOR APPARATUS AND A SYSTEM USING THE SAME

(71) Applicant: UBIQUITY BIOMEDICAL CORPORATION, Taipei (TW)

(72) Inventors: Horng-JI LAI, Taipei (TW); Wan-Ting CHIOU, Taipei (TW); Tzu-Sen YANG, Taipei (TW)

(73) Assignee: UBIQUITY BIOMEDICAL CORPORATION

(21) Appl. No.: 15/398,923

(22) Filed: Jan. 5, 2017

(30) Foreign Application Priority Data
Jun. 1, 2016 (TW) 105117142

(51) Int. Cl. A61B 5/01; A61B 5/00; A61F 7/00

(52) U.S. Cl. CPC A61B 5/01; A61F 7/007; A61B 5/6803; A61B 5/0008; A61B 2560/0223; A61F 2007/0004; A61F 2007/0075

(57) ABSTRACT

A wearable ocular surface temperature monitor apparatus used to measure ocular surface temperatures can be mounted around on eye sockets or can be engaged with glasses. The apparatus comprises a frame and an ocular temperature monitor modular disposed on the frame. The ocular temperature monitor modular further includes a plurality of temperature sensors, a signal processing circuit and a transceiver circuit. The temperature sensors are aligned with ocular surfaces for measuring temperatures. The wireless transceiver circuit reads and records the electrical signals output from the temperature sensors, and outwards wirelessly transmits them by the transceiver circuit.

### CROSS-REFERENCE TO RELATED APPLICATION

This application claims priority from Taiwan Patent Application No. 105117142 filed on Jun. 1, 2016, which are hereby incorporated herein by reference in its entirety.

### BACKGROUND OF THE INVENTION

### 1. Field of the Invention

The invention relates to a wearable ocular surface temperature monitor apparatus, and particularly, to an ocular surface temperature monitor apparatus which is wearable or combined with glasses.

### 2. Description of Related Art

Ocular surface temperatures reflect the current statuses of various muscles and tissue layers of an eyeball, and hence, they can be deemed as an important index representing ocular health or pathological changes. When there is ocular inflammation or ill-sustained accommodation (e.g. spasms) for eye muscles, the ocular surface temperature may rise. For example, pseudo-myopia or myopia happens when the ciliary muscles enter a state of spasm so that the ocular surface temperature gets higher. However, as to a dry-eyes patient, when his eyelids are closed and open, tears flowing into his eyeball surfaces get less so that the ocular surface temperature may fall. Therefore, the increase and decrease in the ocular surface temperature can be used to determine whether an eye is in good health, approaches to have pathological changes or has any pathological changes. In recent years, 3C products have been widely used. Many users spend most of their time for watching images displayed on the 3C products so that they may get myopia or dry-eyes.

U.S. Patent Application No. 2015/0342465 has proposed ocular surface temperature measuring equipment on desk. The volume of such equipment is too big to be portable. Furthermore, only professional technicians can operate the equipment to measure ocular surface temperatures during a quite limited period. The temperature of an ocular surface actually varies with time, circumstance and physiological change. However, the equipment is not suitable to measure the ocular surface temperature for a single user during a long-term period. Thus, it is not applicable to the study and therapy of eye diseases.

Tien-Chun Chang et al. proposed a paper named “Application of digital infrared thermal imaging in determining inflammatory state and follow-up effect of methylprednisolone pulse therapy in patients with Graves' ophthalmopathy” (Graefes Arch Clin Exp Ophthalmol; 2008, 246 vol., pp. 45-49), and indicated that computerized infrared thermal images are used to measure the partial temperatures of a patient with Graves' ophthalmopathy so as to effectively understand and record the inflammatory state of eyeball. Moreover, Jen Hong Tan et al. mentioned that an IR camera is used to collect the surface temperatures of an anterior eyeball in their paper named “Evaluation of topographical variation in ocular surface temperature by function infrared thermography” (Infrared Physics &Technology Volume 54, Issue 6, November 2011, pages 469-47). All such papers employ an IR camera to capture the thermal images of an anterior eyeball. Thus, they have the same problems as the foregoing prior art patent.

In addition, since electrical devices for various use have been miniaturized, wearable or embedded medical (or correcting) apparatus integrated with the electrical devices are widely applied to different technical fields. For example, U.S. Patent Application Nos. 2010/0234717 and 2013/0041245 and PCT International Patent No. 03/0001991 provide a contact lens with an electrical pressure sensor used for measuring intraocular pressure. Moreover, U.S. Patent Application No. 2002/0049389 also puts forth a contact lens combined with a thermal resistor transducer (See paragraphs [0133]-[0134]) for measuring ocular temperatures. These contact lenses have circuits powered by an internal miniature battery (cell) or the induction of external coils. However, the battery or external coils are likely to cause a change in ocular temperatures. That is, the accuracy of the temperature measurement is affected.

In view of above, eye treatment or vision correction is in very need of an apparatus capable of detecting ocular surface temperatures. It can be broadly applied to preventive medicine and correctional health.

### SUMMARY OF THE INVENTION

The present application provides a wearable ocular surface temperature monitor apparatus, and a method for using the same which is wearable for long-term use or combined with glasses so as to record ocular surface temperatures. Therefore, they can be applied to preventive medicine and correctional health.

In view of above aspects, the present invention provides a wearable ocular surface temperature monitor apparatus used to noninvasively measure ocular surface temperatures comprising: a frame including a compartment and at least one opening; and an ocular temperature monitor module including at least one first temperature sensor disposed in the compartment and measuring the ocular surface temperatures through the opening to generate a plurality of electrical signals; and a temperature signal processing circuit receiving and recording the electrical signals, and outwards transmitting wireless signals.

In an embodiment, the temperature signal processing circuit includes a signal processing circuit reading and recording the electrical signals output from the first temperature sensor and a wireless transceiver circuit converting output signals from the signal processing circuit to the wireless signals.

In another embodiment, the apparatus further comprises a thermoelectric cooler driven by the temperature signal processing circuit to reduce the temperatures of air adjacent to an eye.

In another embodiment, the apparatus further comprises a second temperature sensor for measuring the temperatures of skin so as to calibrate or compensate the measured ocular surface temperatures.

In another embodiment, the frame is a detachable part of a spectacle frame.

In another embodiment, the frame is a spectacle frame, the first temperature sensor is disposed within the compartment of the spectacle frame, the temperature signal processing circuit is disposed within a protective shell detachable from the spectacle frame, and the temperature signal processing circuit is in electrical communication with the first temperature sensor through a plurality of metallic contacts.

In another embodiment, the frame further comprises an end cover on which the first temperature sensor is mounted and a spherical shell combined with the end cover and rotatable relative to the frame.

In another embodiment, the temperature signal processing circuit and the first temperature sensor are both disposed with the compartment.

In another embodiment, the apparatus further comprises a lighting device provided on the frame emitting rays or a light pattern when the measured ocular surface temperature is higher or lower that a threshold value.

In another embodiment, the apparatus further comprises a plurality of lenses disposed on the frame and a transparent display device formed on one of the lenses. The transparent display device emits rays, patterns or characters when the measured ocular surface temperature is higher or lower than a threshold value.

the present invention further provides a wearable ocular surface temperature monitor system used to noninvasively measure ocular surface temperatures comprising: a frame including a compartment and at least one opening; an ocular temperature monitor module including at least one first temperature sensor disposed in the compartment and measuring the ocular surface temperatures through the opening to generate a plurality of electrical signals; and a temperature signal processing circuit receiving and recording the electrical signals, and outwards transmitting wireless signals; and a mobile communication apparatus receiving the wireless signals and analyzing and displaying the ocular surface temperatures.

### BRIEF DESCRIPTION OF THE DRAWINGS

In order to sufficiently understand the essence, advantages and the preferred embodiments of the present invention, the following detailed description will be more clearly understood by referring to the accompanying drawings.

FIG. 1 is a schematic diagram of a wearable ocular surface temperature monitor apparatus in accordance with an embodiment of the present application;

FIG. 2 is an exploded view diagram of the wearable ocular surface temperature monitor apparatus as shown in FIG. 1;

FIG. 3A and FIG. 3B are cross-sectional diagrams showing adjustable angles of the spherical shell 1143 relative to the end cover 1144 for the combination of them;

FIG. 4 is a function block diagram of an ocular temperature monitor circuit module in accordance with an embodiment of the present invention;

FIG. 5 is a schematic diagram of a wearable ocular surface temperature monitor apparatus in accordance with another embodiment of the present application; and

FIG. 6 is a schematic diagram of a wearable ocular surface temperature monitor apparatus in accordance with yet another embodiment of the present application.

### DETAILED DESCRIPTION OF THE INVENTION

The following description shows the preferred embodiments of the present invention. The present invention is described below by referring to the embodiments and the figures. Thus, the present invention is not intended to be limited to the embodiments shown, but is to be accorded the principles disclosed herein. Furthermore, that various modifications or changes in light thereof will be suggested to persons skilled in the art and are to be included within the spirit and purview of this application and scope of the appended claims.

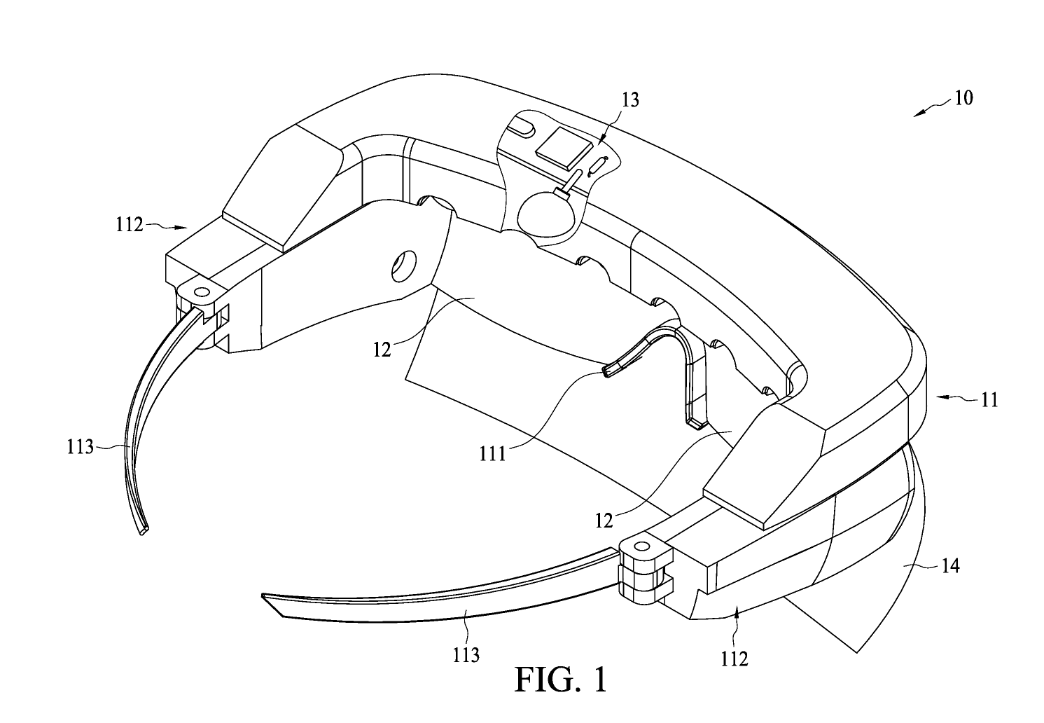

FIG. 1 is a schematic diagram of a wearable ocular surface temperature monitor apparatus in accordance with an embodiment of the present application. A wearable ocular surface temperature monitor apparatus 10 comprises a frame 11, a pair of lenses 12, and an ocular temperature monitor circuit module 13. The ocular temperature monitor circuit module 13 is disposed within the frame 11 for measuring ocular surface temperatures, and it can process and transmit the signals of measured ocular surface temperatures. The frame 11 includes a ring (not shown) for holding the lenses 12, nose pads 111, front temples 112, and rear temples 113. Furthermore, a transparent cover 14 shielding the front side of the lenses 12 so as to avoid the influence of quick air flow on the ocular surface temperatures. Thus, wind is blocked in front of the frame 11 to keep the temperatures from a drop. The embodiment shows a spectacle frame as the frame, but it is not limited to the frame of glasses. The frame can be also mounted on an eye socket or combined with glasses. These embodiments may be included within the scope of the claimed invention.

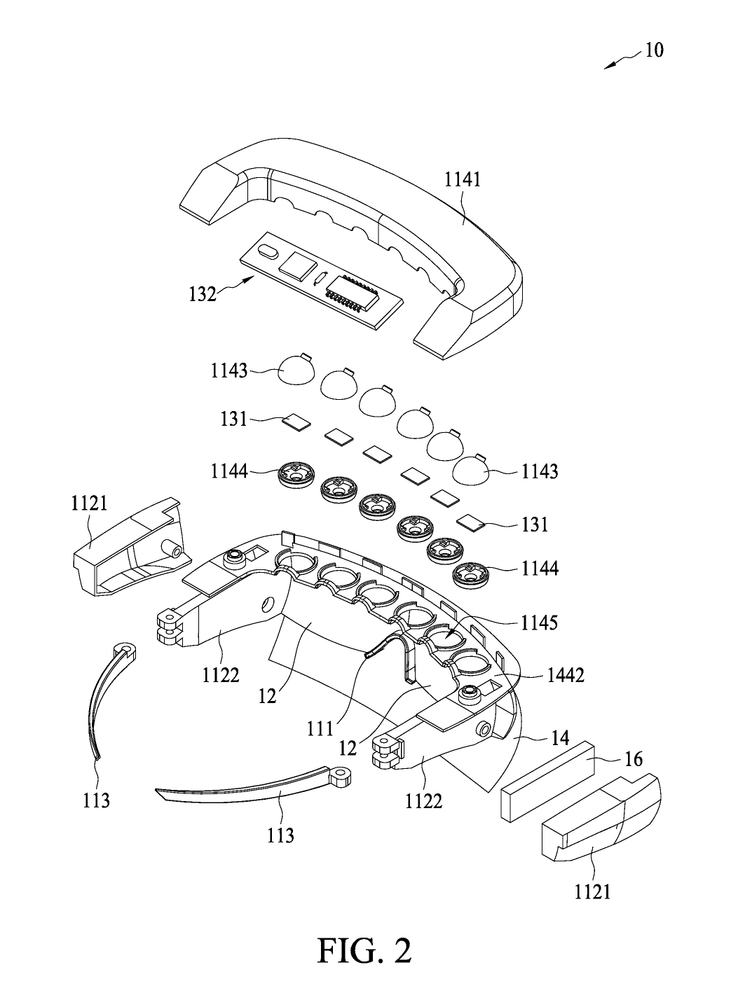

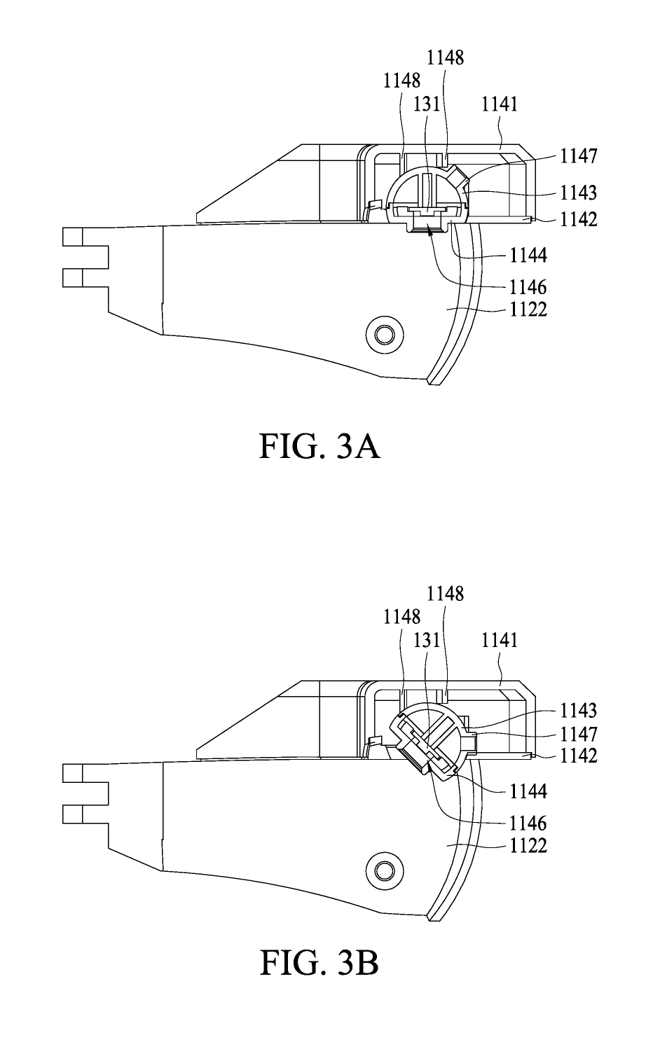

FIG. 2 is an exploded view diagram of the wearable ocular surface temperature monitor apparatus as shown in FIG. 1. An upper cover 1141 and a lower bracket 1142 are disposed on the frame 11, and they are combined with each other. A compartment exists between the upper cover 1141 and the lower bracket 1142, and contains a plurality of temperature sensors 131 (or at least one temperature sensor) and temperature signal processing circuit 132 (a circuit board and electrical components on it as shown in this figure) to protect them. The temperature sensors 131 such as infrared sensors may detect ocular surface temperatures through the effect of thermal radiation, thermal convention or thermal conduction. Each of the temperature sensors 131 is respectively mounted within the end cover 1144. A spherical shell 1143 is combined with the end cover 1144. The combinations are used to adjust the positions of the temperature sensors 131 so that the temperature sensors 131 can be effectively aimed at various portions of an ocular surface to measure temperatures (See FIG. 3A and FIG. 3B). The lower bracket 1142 has a plurality of openings 1145 for the end cover 1144 to be exposed therein. The end cover 1144 can rotate or move relative to the opening 1145.

The front temple 112 includes a lateral cover 1121 and a base 1122. The lateral cover 1121 and the base 1122 are combined with each other, and a compartment containing a thermoelectric cooler 16 is disposed therebetween. When the ocular temperature monitor circuit module 13 detects that the ocular surface temperature is quite higher (e.g. a predetermined value), the thermoelectric cooler 16 is driven to start cooling. The heat of inner air behind the lenses 14 is taken away so that the ocular temperature is indirectly reduced. The thermoelectric cooler 16 may be a thermoelectric cooling module, a thermoelectric cooling chip, a thermoelectric cooler, a Peltier cooler, a Peltier cell, a heat pump. That is, a thermal-electrical material or device capable of converting thermal energy and electrical energy from each other may be included within the scope of the claimed invention.

FIG. 3A and FIG. 3B are cross-sectional diagrams showing adjustable angles of the spherical shell 1143 relative to the end cover 1144 for the combination of them. The spherical shell 1143 is interlocked together with the end cover 1144 on which one of the temperature sensors 131 is mounted. A hole 1146 is located in the middle of the end cover 1144 so that the temperature sensors 131 can detect external temperatures through the hole 1146. For example, it can detect thermal radiation emitting from an ocular surface. A protrusion portion 1147 is provided on the spherical shell 1143, and allows a signal wire and a power wire (not shown) to pass therein and be in connection with the contacts on the board of the temperature signal processing circuit 132. Moreover, the protrusion portion 1147 is also used to limit the rotation angle of the combination of the spherical shell 1143 and the end cover 1144 and prevent the combination from dropping through the opening 1145. Ribs 1148 provided on the upper cover 1141 can constrain the spherical shell 1143 to only have rotational motion. The protrusion portion 1147 is confined between the ribs 1148 and the lower bracket 1142. The spherical shell 1143 has rotational motion with a maximum angle of rotation as shown in FIG. 3A and FIG. 3B. The present application is not limited to the specific embodiment. Any ball joint mechanisms or structures may be included within the scope of the claimed invention.

In the foregoing embodiment, the upper cover 1141 and the lower bracket 1142 are combined with each other, and the temperature sensors 131 and the temperature signal processing circuit 132 are contained in the compartment enclosed by the combination. The whole combination can be mounted on the frame 11 (by screws) or is detachable to the frame 11 by an engagement (e.g. snapping) mechanism. That is, in one of detachable embodiments, the upper cover 1141, the lower bracket 1142 and the ocular temperature monitor circuit module 13 can be deemed as a whole wearable ocular surface temperature monitor apparatus, and the frame 11 and the lenses 12 are optional.

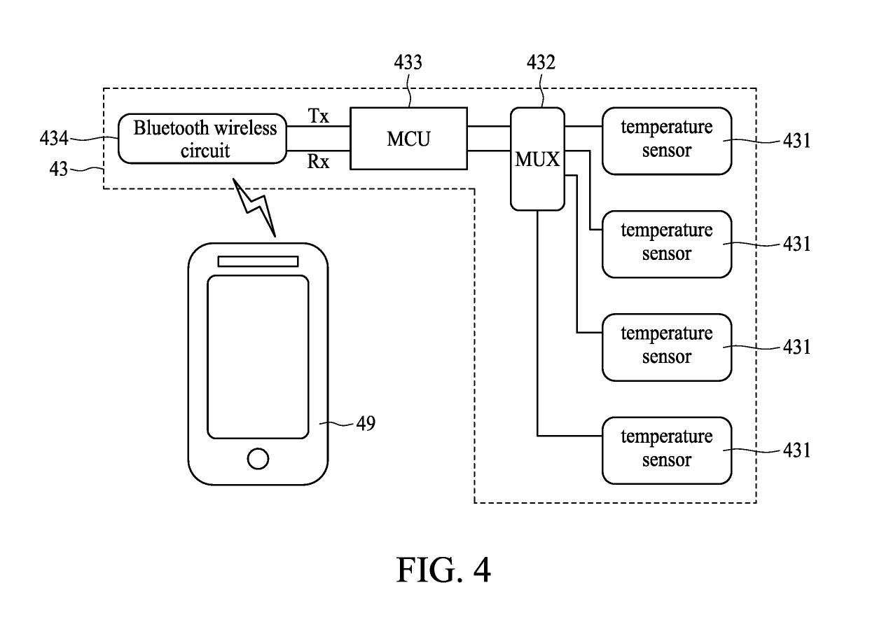

FIG. 4 is a function block diagram of an ocular temperature monitor circuit module in accordance with an embodiment of the present invention. The ocular temperature monitor circuit module 43 comprises a plurality of temperature sensors 431, a multiplexer 432, a micro control unit (MCU) 433 and a Bluetooth wireless circuit 434. The embodiment is an example, and does not limit the scope of the claimed invention of the present application. For example, the Bluetooth wireless circuit 434 can be substituted by other RF (radio frequency) circuits, and the multiplexer 432 and the micro control unit 433 can be integrated into a signal processor or a central process unit (CPU). The temperature sensors 431 aim at various portions of an ocular surface to detect the temperatures of the portions and output analogous signals representing the measured temperatures. The multiplexer 432 selects electrical signals generated from one of the temperature sensors 431 according to the micro control unit 433 and send them to the micro control unit 433. For example, the electrical signals of the temperature sensors 431 are sequentially and repeatedly selected. The micro control unit 433 may read, record and register the foregoing electrical signals or further process (e.g. signal conversion or signal compensation) the signals. The Bluetooth wireless circuit 434 receives the processed signals output from the micro control unit 433 and wirelessly transmits them outwards.

A mobile 49, a computer (not shown), a specific analyzer (not shown) or a mobile communication apparatus (not shown) receives Bluetooth wireless signals or signals complied with another protocol. Then, an App or an analyzing program is used to analyze and compare the forgoing signals representing the ocular temperatures. The measured data of the ocular surface temperatures are displayed by characters or patterns, or it further indicates whether an estimated result is abnormal. The user also can utilize an App of the mobile 49 to set the parameters of the micro control unit 433 or the use modes of the temperature signal processing circuit 132. For example, measuring periods or sampling frequencies can be set.

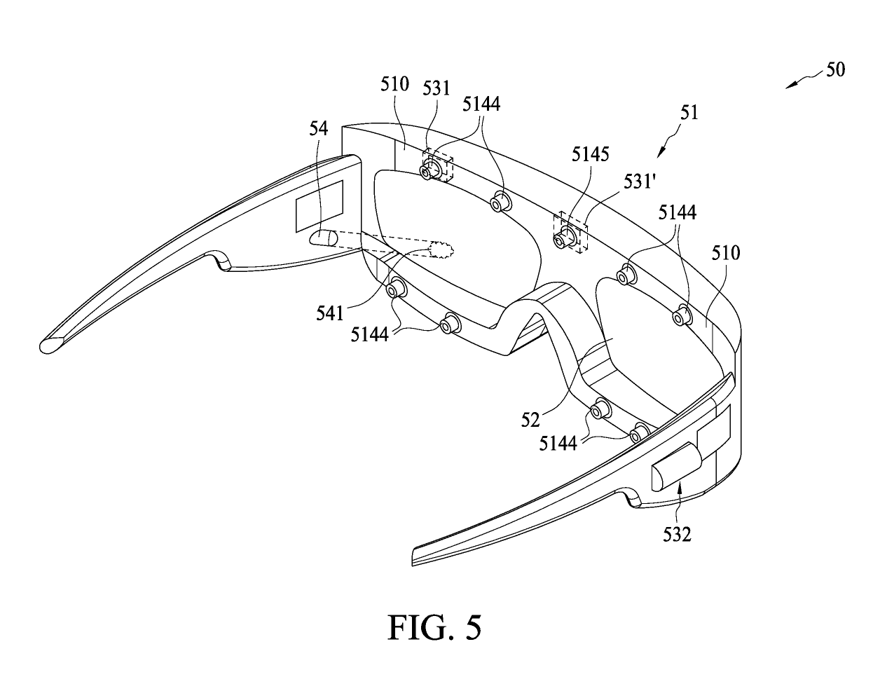

FIG. 5 is a schematic diagram of a wearable ocular surface temperature monitor apparatus in accordance with another embodiment of the present application. As shown in this figure, a wearable ocular surface temperature monitor apparatus 50 comprises a frame 51, a pair of lenses 52, and an ocular temperature monitor circuit module (including a plurality of temperature sensors 531 and a temperature signal processing circuit 532). The ocular temperature monitor circuit module is disposed within the frame 51 for measuring ocular surface temperatures, and it can process and transmit the signals of measured ocular surface temperatures. The temperature signal processing circuit 532 has been described in the foregoing embodiment, and hence there is no further discussion below. In addition, the temperature signal processing circuit 532 may be implemented by an ASIC (Application-Specific IC) device.

The temperature sensors 531 are disposed within the upper and lower rims of the rings 510. The openings of the rims allow rotatable end covers 5144 to extend outward. One of the temperature sensors 531 is fixed to each end cover 5144 (See the corresponding description of the end cover 1144 above). The aiming angle of the temperature sensor 531 is adjustable. The temperature signal processing circuit 532 receives electrical signals generated from temperature sensors through wires (not shown) enclosed within the frame 51 to proceed signal processing and wirelessly transmit them to a mobile or a computer.

In order to avoid the variation of ocular temperatures caused by the changes in body temperatures, a temperature sensor 531′ (also named as a second temperature sensor as being different from temperature sensors 531) is provided on the nose bridge centrally connected the two rings 510 for detecting skin temperatures. The skin temperatures can be deemed as a reference temperature to estimate whether the body temperatures of the user is abnormal (e.g. fever or hypothermia (higher or lower than a default value)). Accordingly, the measured ocular surface temperatures can be compensated or calibrated. Similarly, an opening provided on the nose bridge allows a rotatable end cover 5145 to extend outward. One of the temperature sensors 531 is fixed to each end cover 5144 (See the corresponding description of the end cover 1144 above). The aiming angle of the temperature sensor 531′ is adjustable.

When the ocular surface temperature is quite high or abnormal, the temperature signal processing circuit 532 drives a light emitting device 54 to generate a light image 541 on the lens 52 as an alarm. Thus, the user is noticed to stop watching a screen or leave a high-temperature environment. Furthermore, a transparent soft display device (not shown; or as the light image 541) may be formed on the lens 52. For example, the temperature signal processing circuit 532 drives a soft eBook to display patterns or characteristics as being a warning. Moreover, an organic LED display or liquid crystal display may be formed on the periphery of an visual angle on the lens.

The wearable ocular surface temperature monitor apparatus 50 with an temperature measuring function is suitable for long-term wear during either life time or working time, and does not need to stay at a measurement area for just being closed to a temperature measurement tool. For example, the prior art asked the one under measurement to stand in front of an IR (infrared) measurement gauge. The present invention is suitable for measurement during an activity, sleep or long period so that the accuracy of the syndrome estimation is quite improved.

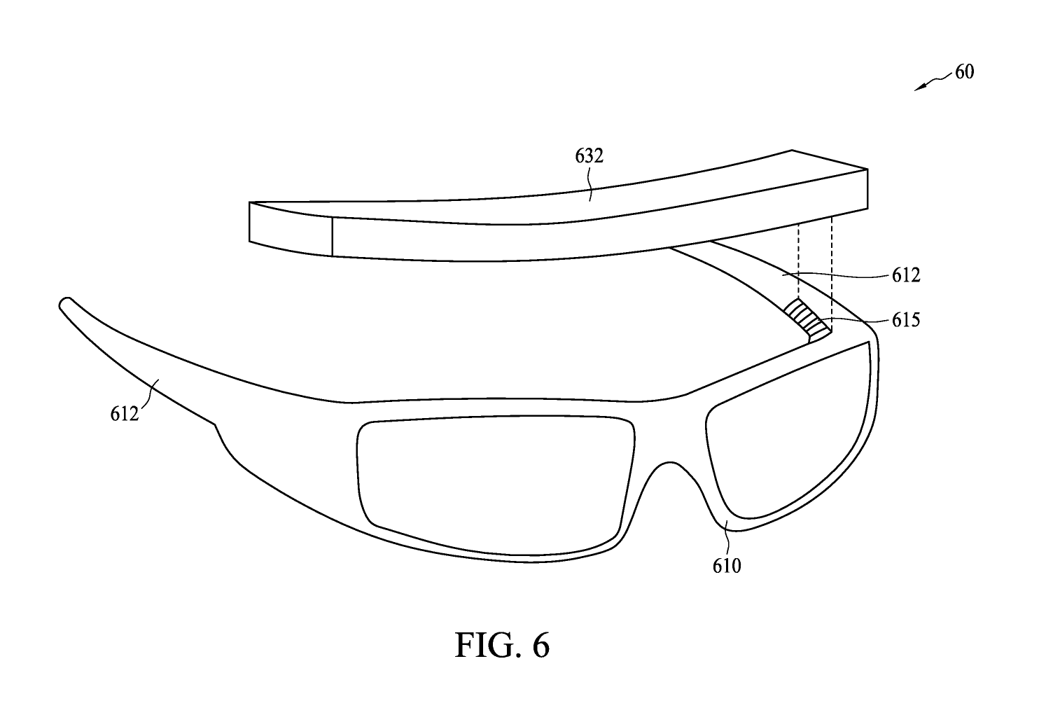

FIG. 6 is a schematic diagram of a wearable ocular surface temperature monitor apparatus in accordance with yet another embodiment of the present application. Compared with FIG. 5, a temperature signal processing circuit 632 of the wearable ocular surface temperature monitor apparatus 60 is disposed within a protection shell (similar to the temperature signal processing circuit 532 as shown in FIG. 5). It is detachable from a frame. A plurality of metallic contacts or golden fingers are provided on a temple 612 for the temperature signal processing circuit 632 to receive the electrical signals of temperature sensors within upper and lower rims of the rings 610.

Wearable ocular surface temperature monitor apparatuses provided by the present application are not only used to monitor the variation in ocular temperatures for myopia and dry-eye, but also are used to inspect and estimate other abnormal conditions (or symptoms) from the variation in ocular temperatures. As shown in FIG. 2, the temperature sensors 131 may aim at different points on the left and right eyeballs. For example, the left three temperature sensors 131 are respectively toward to the inner canthus, middle and outer canthus of the left eye. Of course, more temperature sensors can monitor the temperatures of more points (e.g. points closed to upper and lower eyelids) on an eyeball.

When the upper eyelid (or eyelid) starts to close, the temperature sensors at different locations sequentially detect the temperatures of eyelids because the temperatures of the eyelids are different from those of the eye balls. That is, the measured temperature of an eye ball at the first time point instantaneously changes when the upper eyelid just passes. According the variations in measured temperatures at each point and various time points, it can be determined whether eyelids are closed or the blinking frequency. If the user wearing a wearable ocular surface temperature monitor apparatus has blepharospasm (eyelid jump), his eyelid muscles uncontrollably go into spasm. The twitch may occur in both eyes simultaneously or respectively. The wearable ocular surface temperature monitor apparatus is used to detect the blinking times and frequency of each eye. A threshold value is set for the times or the frequency, and is used to compare with the measured blinking times and frequency. If it is larger than the threshold, blepharospasm may occur in the eye. Of course, a plurality of thresholds may be set according to practical experiences to estimate whether the symptom of blepharospasm is serious or slight.

Furthermore, if the user has thyroid-associated orbitopathy (e.g. Graves ophthalmopathy), its eyelids may not completely close. The wearable ocular surface temperature monitor apparatus is used to detect the temperature distribution from various measured points to determine whether such symptom or condition happens. That is, the temperatures measured at eyelid-uncovered points are different from the temperatures measured at points on eyelids.

In view of above, the temperature sensors can monitor the temperatures of various points on eyeballs. If the use fast turns eye balls or has cross-eye, the direction or trend for the temperature changes in various points can be used to determine whether an eyeball turns or its rotation direction. That is, maximum (or minimum) temperatures may sequentially occur in adjacent points on an eyeball during a measuring period.

Tourette syndrome (TS) is a common neuropsychiatric disorder with onset in childhood, characterized by multiple motor tics and at least one vocal (phonic) tic. Some common tics are cross-eye, fast eye rotation, or eye blinking. Therefore, if a user is considered as a Tourette patient, the wearable ocular surface temperature monitor apparatus can be used to long-term monitor his eye motion, and estimate the Tourette syndrome according to predetermined conditions.

The foregoing embodiments of the invention have been presented for the purpose of illustration. Although the invention has been described by certain preceding examples, it is not to be construed as being limited by them. They are not intended to be exhaustive, or to limit the scope of the invention. Modifications, improvements and variations within the scope of the invention are possible in light of this disclosure.

What is claimed is:

1. A wearable ocular surface temperature monitor apparatus used to noninvasively measure ocular surface temperatures comprising:
a frame including a compartment and at least one opening; and
an ocular temperature monitor module including:
at least one first temperature sensor disposed in the compartment and measuring the ocular surface temperatures through the opening to generate a plurality of electrical signals; and
a temperature signal processing circuit receiving and recording the electrical signals, and outwards transmitting wireless signals.

2. The wearable ocular surface temperature monitor apparatus according to claim 1, wherein the temperature signal processing circuit includes:
a signal processing circuit reading and recording the electrical signals output from the first temperature sensor; and
a wireless transceiver circuit converting output signals from the signal processing circuit to the wireless signals.

3. The wearable ocular surface temperature monitor apparatus according to claim 1, further comprising a thermoelectric cooler driven by the temperature signal processing circuit to reduce the temperatures of air adjacent to an eye.

4. The wearable ocular surface temperature monitor apparatus according to claim 1, further comprising a second temperature sensor for measuring the temperatures of skin so that the measured ocular surface temperatures are calibrated or compensated.

5. The wearable ocular surface temperature monitor apparatus according to claim 1, wherein the frame is a detachable part of a spectacle frame.

6. The wearable ocular surface temperature monitor apparatus according to claim 1, wherein the frame is a spectacle frame, the first temperature sensor is disposed within the compartment of the spectacle frame, the temperature signal processing circuit is disposed within a protective shell detachable from the spectacle frame, and the temperature signal processing circuit is in electrical communication with the first temperature sensor through a plurality of metallic contacts.

7. The wearable ocular surface temperature monitor apparatus according to claim 1, wherein the frame further includes:
an end cover on which the first temperature sensor is mounted; and
a spherical shell combined with the end cover and rotatable relative to the frame.

8. The wearable ocular surface temperature monitor apparatus according to claim 1, wherein the temperature signal processing circuit and the first temperature sensor are both disposed with the compartment.

9. The wearable ocular surface temperature monitor apparatus according to claim 1, further comprising a lighting device provided on the frame emitting rays or a light pattern when the measured ocular surface temperature is higher or lower than a threshold value.

10. The wearable ocular surface temperature monitor apparatus according to claim 1, further comprising a plurality of lenses disposed on the frame and a transparent display device formed on one of the lenses; wherein the transparent display device emitting rays, a light pattern or characters when the measured ocular surface temperature is higher or lower than a threshold value.

11. The wearable ocular surface temperature monitor apparatus according to claim 1, wherein the number of the at least one first temperature sensor is plural, and the first temperature sensors respectively measure surface temperatures of a plurality of points on eyeballs and eyelids.

12. The wearable ocular surface temperature monitor apparatus according to claim 1, wherein the number of the at least one first temperature sensor is plural, and the first temperature sensors continuously and respectively measure surface temperatures of a plurality of points on eyeballs.

13. A wearable ocular surface temperature monitor system used to noninvasively measure ocular surface temperatures comprising:
a frame including a compartment and at least one opening;
an ocular temperature monitor module including:
at least one first temperature sensor disposed in the compartment and measuring the ocular surface temperatures through the opening to generate a plurality of electrical signals; and
a temperature signal processing circuit receiving and recording the electrical signals, and outwards transmitting wireless signals; and
a mobile communication apparatus receiving the wireless signals and analyzing and displaying the ocular surface temperatures.

14. The wearable ocular surface temperature monitor system according to claim 13, wherein the temperature signal processing circuit includes:
a signal processing circuit reading and recording the electrical signals output from the first temperature sensor; and
a wireless transceiver circuit converting output signals from the signal processing circuit to the wireless signals.

15. The wearable ocular surface temperature monitor system according to claim 13, further comprising a thermoelectric cooler driven by the temperature signal processing circuit to reduce the temperatures of air adjacent to an eye.

16. The wearable ocular surface temperature monitor system according to claim 13, further comprising a second temperature sensor for measuring the temperatures of skin so that the measured ocular surface temperatures are calibrated or compensated.

17. The wearable ocular surface temperature monitor system according to claim 13, wherein the frame is a detachable part of a spectacle frame.

18. The wearable ocular surface temperature monitor system according to claim 13, wherein the frame is a spectacle frame, the first temperature sensor is disposed within the compartment of the spectacle frame, the temperature signal processing circuit is disposed within a protective shell detachable from the spectacle frame, and the temperature signal processing circuit is in electrical communication with the first temperature sensor through a plurality of metallic contacts.

19. The wearable ocular surface temperature monitor system according to claim 13, wherein the frame further includes:
an end cover on which the first temperature sensor is mounted; and
a spherical shell combined with the end cover and rotatable relative to the frame.

20. The wearable ocular surface temperature monitor system according to claim 13, wherein the temperature signal processing circuit and the first temperature sensor are both disposed with the compartment.

21. The wearable ocular surface temperature monitor system according to claim 13, further comprising a lighting device provided on the frame emitting rays or a light pattern when the measured ocular surface temperature is higher or lower than a threshold value.

22. The wearable ocular surface temperature monitor system according to claim 13, further comprising a plurality of lenses disposed on the frame and a transparent display device formed on one of the lenses; wherein the transparent display device emitting rays, a light pattern or characters when the measured ocular surface temperature is higher or lower than a threshold value.

23. The wearable ocular surface temperature monitor system according to claim 13, wherein the number of the at least one first temperature sensor is plural, and the first temperature sensors respectively measure surface temperatures of a plurality of points on eyeballs and eyelids.

24. The wearable ocular surface temperature monitor system according to claim 13, wherein the number of the at least one first temperature sensor is plural, and the first temperature sensors continuously and respectively measure surface temperatures of a plurality of points on eyeballs.
